(19) United States
(12) Patent Application Publication
(10) Pub. No.: US 2023/0256269 A1
(43) Pub. Date: Aug. 17, 2023

(54) ANTI-VIRAL BREATHING AND OXYGEN SUPPLYING APPARATUS

(71) Applicants: Jay Clarke Hanan, Ontario, CA (US); Bamidele Herbert Ali, Evanston, IL (US)

(72) Inventors: Jay Clarke HANAN, Ontario, CA (US); Bamidele Herbert ALI, Evanston, IL (US)

(21) Appl. No.: 18/003,761

(22) PCT Filed: Jun. 30, 2021
(86) PCT No.: PCT/US2021/039969

§ 371 Date: Dec. 29, 2022

Related U.S. Application Data
(60) Provisional application No. 63/046,599, filed on Jun. 30, 2020.

(51) Int. Cl. A62B 7/10; A61M 16/08; A61L 9/20; A62B 23/02

(52) U.S. Cl. CPC A62B 7/10; A61L 9/20; A61M 16/0875; A62B 23/02; A61L 2209/14; A61M 2202/0208

(57) ABSTRACT

A user-wearable breathing apparatus is disclosed. Air may be passed through an air irradiating chamber coupled to an ultraviolet light source and configured to irradiate the air irradiating chamber with ultraviolet light. An ultraviolet-opaque cover substantially surrounding the user-wearable breathing apparatus may be provided. A hose may be passed through an opening in the ultraviolet-opaque cover to direct processed air to the user. The hose may be configured to attach to an optional head-wearable breathing assembly. In some embodiments, the air may be passed through the apparatus in response to the user’s breathing, while in other embodiments, fans are used. In other embodiments, the oxygen content of the processed air may be increased by techniques such as nitrogen scrubbing and injecting oxygen into the air while it is being processed.

### PRIORITY

This application claims the benefit of and priority to U.S. Provisional Application No. 63/046,599, filed Jun. 30, 2020, which is incorporated in its entirety herein.

### FIELD

The present disclosure relates to biologically protective equipment. More particularly, the present disclosure relates to user-wearable anti-viral and oxygen supplying breathing apparatuses.

### BACKGROUND

The recent COVID-19 pandemic has caused a great deal of tragedy worldwide. Millions have died. Tens of millions more have lost their livelihood, been rendered homeless, or been forced to struggle to acquire or maintain the basic necessities of everyday life. Economically, the consequences have also been severe. The large upsurge in lost jobs has caused a decrease in consumer spending, which exacerbates the job loss problem. The closing of many businesses and factories in so many countries has greatly disrupted the global supply chains creating shortages in many goods and commodities.

From a public health perspective, the results have been mixed. In the US, there was no effective response at the federal level, so states were on their own to cope as best as they could. Around the world, the results have also been mixed with some countries acting swiftly and surely, typically with better results, and others not reacting or faring so well. With the development of vaccines, the general trend is positive in most places where they are available.

In many communities, many people have been in a state of lockdown for more than a year with only limited ability to go out for necessities like food, medications, and doctor visits. Although sometimes controversial, public health officials in many places have mandated the wearing of face masks in public under certain circumstances. There are many varieties of acceptable protective gear for the public to choose from. Since none are perfect, there is room to develop better alternatives.

### BRIEF DESCRIPTION OF DRAWINGS

The above, and other, aspects, features, and advantages of several embodiments of the present disclosure will be more apparent from the following description as presented in conjunction with the following several figures of the drawings.

FIG. 1 is a conceptual diagram of a system employing a user-wearable breathing apparatus in accordance with an embodiment of the disclosure;

FIG. 2A is a conceptual diagram of a system employing a user-wearable breathing apparatus in accordance with an embodiment of the disclosure;

FIG. 2B is a conceptual diagram of a system employing a user-wearable breathing apparatus in accordance with an embodiment of the disclosure;

FIG. 2C is a conceptual diagram of a system employing a user-wearable breathing apparatus in accordance with an embodiment of the disclosure;

FIG. 3 is a conceptual diagram of a system employing a user-wearable breathing apparatus in accordance with an embodiment of the disclosure;

FIG. 4 is a conceptual diagram of a system employing a user-wearable breathing apparatus in accordance with an embodiment of the disclosure;

FIG. 5 is a conceptual diagram of a system employing a user-wearable breathing apparatus in accordance with an embodiment of the disclosure;

FIG. 6 is a conceptual diagram of a system employing a user-wearable breathing apparatus in accordance with an embodiment of the disclosure;

FIG. 7 is a conceptual diagram of a user-wearable breathing apparatus in accordance with an embodiment of the disclosure;

FIG. 8 is a conceptual diagram of a user-wearable breathing apparatus in accordance with an embodiment of the disclosure;

FIG. 9 is a flowchart depicting a process for operating a user-wearable breathing apparatus in accordance with an embodiment of the disclosure; and

FIG. 10 is a flowchart depicting a process for constructing a user-wearable breathing apparatus in accordance with an embodiment of the disclosure.

Corresponding reference characters indicate corresponding components throughout the several figures of the drawings. Elements in the several figures are illustrated for simplicity and clarity and have not necessarily been drawn to scale. For example, the dimensions of some of the elements in the figures might be emphasized relative to other elements for facilitating understanding of the various presently disclosed embodiments. In addition, common, but well-understood, elements that are useful or necessary in a commercially feasible embodiment are often not depicted in order to facilitate a less obstructed view of these various embodiments of the present disclosure.

### DETAILED DESCRIPTION

In response to the situations described above, a user-wearable anti-viral breathing apparatus is disclosed. An air irradiating chamber (AIC) may be employed to efficiently irradiate external air with ultraviolet light to kill viruses before directing it to the wearer for breathing. In some embodiments, various filtering techniques may be employed internal to, before, and/or after the air irradiating chamber to further purify the air passing through it. In other embodiments, a nitrogen scrubber may be employed internal to, before, and/or after the air irradiating chamber to increase the oxygen content of the processed air. In many embodiments, the apparatus may be worn in a variety of ways, like, for example, strapped to the wearer’s chest inside or outside of a shirt-like garment, in a backpack, a bag, a sack, a shoulder bag, attached to a belt, etc. In yet more embodiments, the user may employ any of a variety of facemasks, faceplates, helmets, or the like coupled to the apparatus. In alternative embodiments, the apparatus is configured to direct air toward a user’s face providing a dynamic pocket of processed air to breathe while pushing aside external airborne contaminants. In various embodiments, the air flowing through the air irradiating chamber results from the user’s breathing, while in some alternative embodiments, a fan, a pump, an impeller, a propeller, or other air moving device may be used to move the air.

Aspects of the present disclosure may be embodied as an apparatus, system, method, or computer program product. Accordingly, aspects of the present disclosure may take the form of an entirely hardware embodiment, an entirely software embodiment (including firmware, resident software, micro-code, or the like) or an embodiment combining software and hardware aspects that may all generally be referred to herein as a “function,” “module,” “apparatus,” or “system.” Furthermore, aspects of the present disclosure may take the form of a computer program product embodied in one or more non-transitory computer-readable storage media storing computer-readable and/or executable program code. Many of the functional units described in this specification have been labeled as functions, in order to emphasize their implementation independence more particularly. For example, a function may be implemented as a hardware circuit comprising custom VLSI circuits or gate arrays, off-the-shelf semiconductors such as logic chips, transistors, or other discrete components. A function may also be implemented in programmable hardware devices such as via field programmable gate arrays, programmable array logic, programmable logic devices, or the like.

Functions may also be implemented at least partially in software for execution by various types of processors. An identified function of executable code may, for instance, comprise one or more physical or logical blocks of computer instructions that may, for instance, be organized as an object, procedure, or function. Nevertheless, the executables of an identified function need not be physically located together but may comprise disparate instructions stored in different locations which, when joined logically together, comprise the function and achieve the stated purpose for the function.

Indeed, a function of executable code may include a single instruction, or many instructions, and may even be distributed over several different code segments, among different programs, across several storage devices, or the like. Where a function or portions of a function are implemented in software, the software portions may be stored on one or more computer-readable and/or executable storage media. Any combination of one or more computer-readable storage media may be utilized. A computer-readable storage medium may include, for example, but not limited to, an electronic, magnetic, optical, electromagnetic, infrared, or semiconductor system, apparatus, or device, or any suitable combination of the foregoing, but would not include propagating signals. In the context of this document, a computer readable and/or executable storage medium may be any tangible and/or non-transitory medium that may contain or store a program for use by or in connection with an instruction execution system, apparatus, processor, or device.

Computer program code for carrying out operations for aspects of the present disclosure may be written in any combination of one or more programming languages, including an object-oriented programming language such as Python, Java, Smalltalk, C++, C#, Objective C, or the like, conventional procedural programming languages, such as the “C” programming language, scripting programming languages, assembly languages, and/or other similar programming languages. The program code may execute partly or entirely on one or more of a user’s computer and/or on a remote computer or server over a data network or the like.

A component, as used herein, comprises a tangible, physical, non-transitory device. For example, a component may be implemented as a hardware logic circuit comprising custom VLSI circuits, gate arrays, or other integrated circuits; off-the-shelf semiconductors such as logic chips, transistors, or other discrete devices; and/or other mechanical or electrical devices. A component may also be implemented in programmable hardware devices such as field programmable gate arrays, programmable array logic, programmable logic devices, or the like. A component may comprise one or more silicon integrated circuit devices (e.g., chips, die, die planes, packages) or other discrete electrical devices, in electrical communication with one or more other components through electrical lines of a printed circuit board (PCB) or the like. Each of the functions and/or modules described herein, in certain embodiments, may alternatively be embodied by or implemented as a component.

A circuit, as used herein, comprises a set of one or more electrical and/or electronic components providing one or more pathways for electrical current. In certain embodiments, a circuit may include a return pathway for electrical current, so that the circuit is a closed loop. In another embodiment, however, a set of components that does not include a return pathway for electrical current may be referred to as a circuit (e.g., an open loop). For example, an integrated circuit may be referred to as a circuit regardless of whether the integrated circuit is coupled to ground (as a return pathway for electrical current) or not. In various embodiments, a circuit may include a portion of an integrated circuit, an integrated circuit, a set of integrated circuits, a set of non-integrated electrical and/or electrical components with or without integrated circuit devices, or the like. In one embodiment, a circuit may include custom VLSI circuits, gate arrays, logic circuits, or other integrated circuits; off-the-shelf semiconductors such as logic chips, transistors, or other discrete devices; and/or other mechanical or electrical devices. A circuit may also be implemented as a synthesized circuit in a programmable hardware device such as field programmable gate array, programmable array logic, programmable logic device, or the like (e.g., as firmware, a netlist, or the like). A circuit may comprise one or more silicon integrated circuit devices (e.g., chips, die, die planes, packages) or other discrete electrical devices, in electrical communication with one or more other components through electrical lines of a printed circuit board (PCB) or the like. Each of the functions and/or modules described herein, in certain embodiments, may be embodied by or implemented as a circuit.

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to”, unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Further, as used herein, reference to reading, writing, loading, storing, buffering, and/or transferring data can include the entirety of the data, a portion of the data, a set of the data, and/or a subset of the data. Likewise, reference to reading, writing, loading, storing, buffering, and/or transferring non-host data can include the entirety of the non-host data, a portion of the non-host data, a set of the non-host data, and/or a subset of the non-host data.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

Aspects of the present disclosure are described below with reference to schematic flowchart diagrams and/ or schematic block diagrams of methods, apparatuses, systems, and computer program products according to embodiments of the disclosure. It will be understood that each block of the schematic flowchart diagrams and/or schematic block diagrams, and combinations of blocks in the schematic flowchart diagrams and/or schematic block diagrams, can be implemented by computer program instructions. These computer program instructions may be provided to a processor of a computer or other programmable data processing apparatus to produce a machine, such that the instructions, which execute via the processor or other programmable data processing apparatus, create means for implementing the functions and/or acts specified in the schematic flowchart diagrams and/or schematic block diagrams block or blocks.

It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated figures. Although various arrow types and line types may be employed in the flowchart and/or block diagrams, they are understood not to limit the scope of the corresponding embodiments. For instance, an arrow may indicate a waiting or monitoring period of unspecified duration between enumerated steps of the depicted embodiment.

In the following detailed description, reference is made to the accompanying drawings, which form a part thereof. The foregoing summary is illustrative only and is not intended to be in any way limiting. In addition to the illustrative aspects, embodiments, and features described above, further aspects, embodiments, and features will become apparent by reference to the drawings and the following detailed description. The description of elements in each figure may refer to elements of proceeding figures. Like numbers may refer to like elements in the figures, including alternate embodiments of like elements.

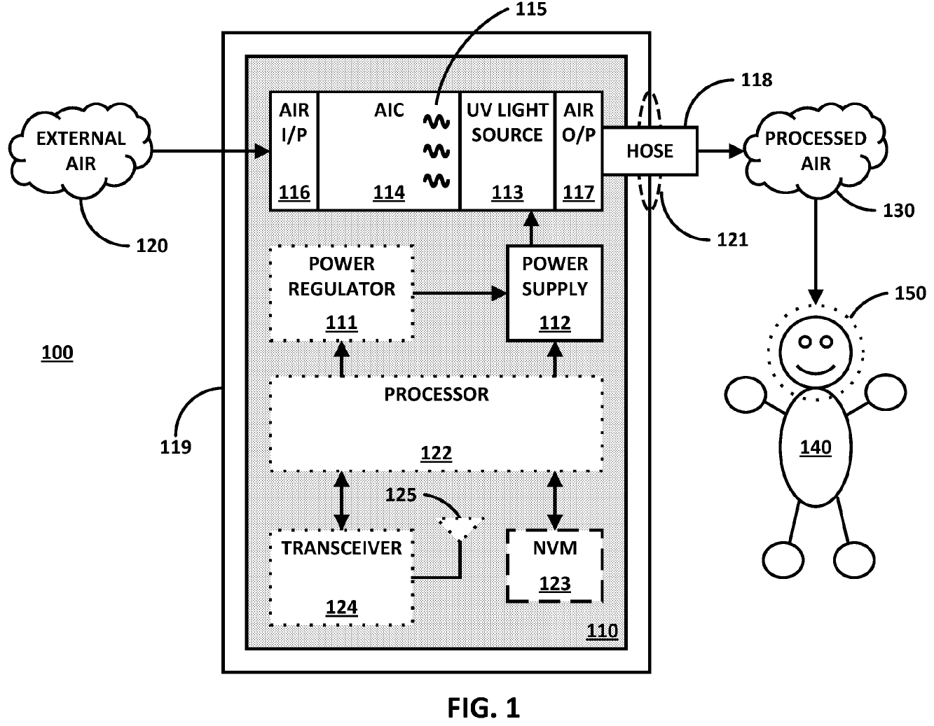

Referring to FIG. 1, a conceptual diagram of a system employing a user-wearable breathing apparatus in accordance with an embodiment of the disclosure is shown. System 100 may comprise apparatus 110, which may further comprise optional power regulator 111, power supply 112, ultraviolet light source 113, air irradiating chamber 114, air input 116, air output 117, hose 118, ultraviolet-opaque cover 119, opening 121, and a number of optional functions like processor 122, non-volatile memory 123, communications transceiver 124, and antenna 125. Optional power regulator 111 may be electrically coupled to power supply 112, which may be further electrically coupled to ultraviolet light source 113. Ultraviolet light source 113 and air irradiating chamber 114 may be coupled to apparatus 110 directly and/or indirectly and/or to each other and configured so that ultraviolet light 115 from ultraviolet light source 113 may irradiate air passing through air irradiating chamber 114. In some embodiments, ultraviolet light source 113 irradiates the airflow in air irradiation chamber 114 substantially in parallel to the direction of airflow. In other embodiments, the irradiation may be substantially perpendicular to the airflow, while in further embodiments, the irradiation may occur at an angle to airflow.

Air irradiating chamber 114 may further comprise air input 116 and air output 117. A first end of a hose 118 may be coupled to air output 117. The apparatus 110 may further comprise an ultraviolet-opaque covering 119, which may protect the user and others from irradiation by ultraviolet light source 113 and/or provide an anchor for straps, etc., for wearing the apparatus 110. Ultraviolet-opaque covering 119 may be configured to substantially enclose the rest of apparatus 110 during operation but may be opened to allow access when apparatus 110 is not in use. Ultraviolet-opaque covering 119 may further comprise an opening 121 through which hose 118 may pass. In various embodiments, the ultraviolet-opaque covering may be a backpack, a shirt-like garment, a carrying bag, a cloth bag, an ultraviolet-opaque box, an ultraviolet-opaque bottle, another ultraviolet-opaque container, and/or a coating of ultraviolet-opaque paint. In certain embodiments, ultraviolet-opaque covering 119 may also be porous and function as a filter and be treated with anti-viral and/or anti-microbial substances. In other embodiments, ultraviolet-opaque covering 119 may have openings that may allow external air 120 access to air input 116.

In operation, system 100 may draw external air 120 through the ultraviolet-opaque cover 119, through air input 116, and into air irradiating chamber 114. Air irradiating chamber 114 may be ultra-violet transparent. As the air traverses the air irradiating chamber 114, it may be irradiated by ultraviolet light 115 from ultraviolet light source 113. The intensity of the ultraviolet light from ultraviolet light source 113 may be greater than or substantially equal to 50 micro-Watts per square centimeter (µW/cm²), and its wavelength may be between substantially 100 nanometers and substantially 280 nanometers.

The air is drawn out of air irradiating chamber 114 through air output 117 and into hose 118. The processed air 130 may be directed to user 140 for breathing. User 140 may be wearing an optional head-wearable breathing assembly 150 like, for example, a facemask, a faceplate, a helmet, or the like that may be coupled to a second end of hose 118. The second end of hose 118 may be configured to couple to head-wearable breathing assembly 150. Alternatively, the processed air 130 from the second end of hose 118 may be directed to the face of user 140 to create a dynamic pocket of processed air 130 to breathe while pushing away any contaminants in the surrounding external air 120.

In some embodiments, the air irradiating chamber 114 may comprise a maze-like structure. In other embodiments, the air irradiating chamber 114 may comprise a porous anti-viral material, at least in part, like, for example, Zoono® or graphene (not shown). In yet other embodiments, a combination of a maze-like structure filled with porous anti-viral material may be used. In many embodiments, air irradiating chamber 114 be empty, while additional filtering (not shown) may be applied before, during, and/or after the external air 120 passes through the apparatus 110. Such additional filtering may be constructed at least in part by an anti-microbial material such as activated charcoal or nano-silver or the like to further purify the air.

In yet more embodiments, an optional nitrogen scrubber (not shown) may be applied before, during, and/or after the external air 120 passes through apparatus 110. This increases the portion of oxygen in processed air 130. In some alternative embodiments, air irradiating chamber 114 may further comprise an optional gas port (not shown). Such an optional gas port may be used, for example, to inject oxygen into air irradiation chamber 114, couple to a trachea catheter, etc. In some more embodiments, adjustable valves may be present with the gas port, air input 116, and air output 117, allowing the air flows to be adjusted manually or automatically by optional processor 122.

In many embodiments, power supply 112 may comprise or be coupled to an energy storage device (not shown), like, for example, a battery, a capacitor, a super-capacitor, etc. The energy storage device may be rechargeable and may be charged by optional power regulator 111. In some embodiments, optional power regulator 111 may be configured to couple to an external power supply (not shown), like, for example, power mains directly (or indirectly through a battery charging device) and may be used to recharge the energy storage device and/or power ultraviolet light source 113 directly. In other embodiments, optional power regulator 111 may be configured to couple to an energy harvesting device (not shown) such as a solar panel, a piezoelectric and/or electrostatic and/or electromagnetic and/or another kinetic-to-electrical energy converter, an ambient radiation-to-electrical converter, a thermoelectric generator, and/or another temperature-difference-to-electrical energy converter, or the like, and may be used to recharge the energy storage device and/or power ultraviolet light source 113 directly.

In some other embodiments, optional power regulator 111 and power supply 112 may be coupled to optional processor 122. Optional processor 122 may execute machine instructions that may be persistently stored in optional non-volatile machine-readable memory 123. Optional processor 122 may monitor the operational state of optional power regulator 111 and power supply 112. In yet other embodiments, various sensors (not shown) may be present and coupled to optional processor 122, which may allow it to monitor the operational state of other aspects of apparatus 110. In still more embodiments, optional processor 122 may be coupled to various controls on some components (not shown), like, for example, a fan that may be turned on or off or have its speed adjusted, etc.

In various embodiments, optional communications transceiver 124 may be coupled to optional processor 122, which may be coupled to optional antenna 125. Optional communications transceiver 124 may be configured to wirelessly communicate with an external system, and optional processor 122 may communicate with the external system through the optional communications transceiver 124. User 140 or another user may control the apparatus 110 wirelessly by sending instructions to optional processor 122. Such instructions may, for example, be to report the operational state of apparatus 110, control various parameters (e.g., the intensity of the ultraviolet light from ultraviolet light source 113, the speed of a fan, etc.), and update the machine instructions that may be persistently stored in optional non-volatile machine-readable memory 123.

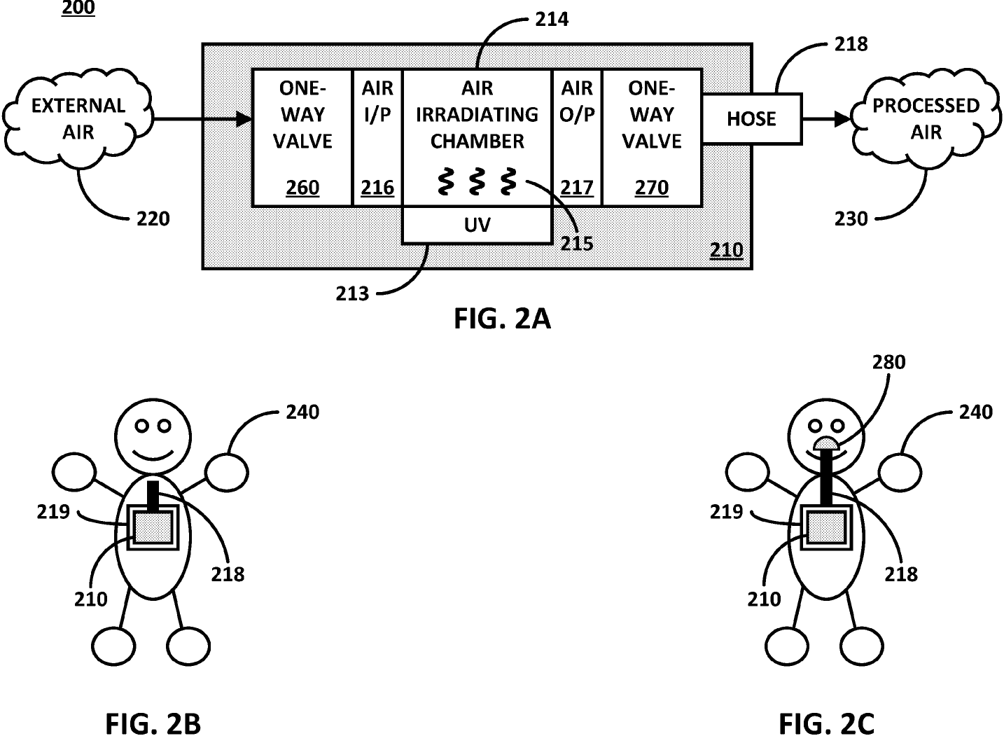

Referring to FIG. 2A, a conceptual diagram of a system employing a user-wearable breathing apparatus in accordance with an embodiment of the disclosure is shown. System 200 comprises apparatus 210, which further comprises ultraviolet light source 213, air irradiating chamber 214, air input 216, air output 217, hose 218, and one-way valves 260 and 270. Certain components which may be present in various embodiments of apparatus 210, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Ultraviolet light source 213 and air irradiating chamber 214 may be coupled to apparatus 210 such that ultraviolet light from ultraviolet light source 213 may irradiate air passing through air irradiating chamber 214 with ultraviolet light 215. While shown irradiating the air flowing through air irradiating chamber 214 substantially perpendicular to the airflow, in other embodiments, the airflow may be irradiated substantially in parallel with or at an angle to the airflow. Air irradiating chamber 214 may further comprise air input 216 and air output 217. Air input 216 may be coupled to a first one-way valve 260, and air output 217 may be coupled to a second one-way valve 270. In some embodiments, one-way valves 260 and 270 may be identical. In other embodiments, two different types of one-way valves may be used. A first end of hose 218 may be coupled to the second one-way valve 270.

In operation, system 200 may draw external air 220 through an ultraviolet-opaque cover (not shown), through the first one-way valve 260, through air input 216, and into air irradiating chamber 214. Air irradiating chamber 214 may be ultra-violet transparent. As the air traverses the air irradiating chamber 214, it may be irradiated by ultraviolet light from ultraviolet light source 213. The air is drawn out of air irradiating chamber 214 through air output 217, through second one-way valve 270, and into the first end of hose 218. The processed air 230 may be directed from a second end of hose 218 to the user (not shown) for breathing.

In some embodiments, the air irradiating chamber 214 may comprise a maze-like structure. In other embodiments, the air irradiating chamber 214 may comprise porous anti-viral material, at least in part, like, for example, Zoono® or graphene (not shown). In yet more embodiments, a combination of a maze-like structure filled with porous anti-viral material. In some alternative embodiments, additional filtering (not shown) may be applied before, during, and/or after the external air 220 passes through apparatus 210. Such additional filtering may be constructed at least in part by an anti-microbial material such as activated charcoal or nano-silver or the like to further purify the air. In yet more embodiments, a nitrogen scrubber (not shown) may be applied before, during, and/or after the external air 220 passes through apparatus 210.

Referring to FIG. 2B, a conceptual diagram of a system employing a user-wearable breathing apparatus in accordance with an embodiment of the disclosure is shown. The system may comprise apparatus 210, which may further comprise hose 218 and ultraviolet-opaque cover 219 as described with respect to FIG. 2A above, though many elements have been omitted for clarity of presentation. Also illustrated in the figure is user 240. In some embodiments, hose 218 is positioned so that the processed air 230 from apparatus 210 is directed towards the face of user 240 to create a dynamic pocket of processed air to breathe while pushing away any contaminants in external air 220.

Apparatus 210 may be coupled to the upper torso of user 240. Apparatus 210 may be coupled in a variety of ways, like, for example, straps going around the upper torso of user 240 (not shown) or coupled to a shirt-like garment (not shown) worn by user 240, etc. First and second one-way valves 260 and 270 (not shown) may be configured to draw air through apparatus 210 and hose 218 in response to the motion of breathing by user 240. This may save considerable battery life that would otherwise be unavailable to the ultraviolet light source 213 (not shown).

Referring to FIG. 2C, a conceptual diagram of a system employing a user-wearable breathing apparatus in accordance with an embodiment of the disclosure is shown. The system may comprise apparatus 210, which may further comprise hose 218 and ultraviolet-opaque cover 219 as described with respect to FIG. 2A and FIG. 2B above, though many elements have been omitted for clarity of presentation. Also illustrated in the figure are user 240 and head-wearable breathing assembly 280. The head-wearable breathing assembly 280 may be coupled to the second end of hose 218. User 240 may be wearing head-wearable breathing assembly 280, which may comprise, for example, a facemask (shown), a faceplate (not shown), a helmet (not shown), or the like.

Apparatus 210 may be coupled to the upper torso of user 240. Apparatus 210 may be coupled in a variety of ways, like, for example, straps going around the upper torso of user 240 (not shown) or coupled to a shirt-like garment (not shown) worn by user 240, etc. First and second one-way valves 260 and 270 (not shown) may be configured to draw air through apparatus 210 and hose 218 in response to breathing by user 240. This may save considerable battery life that would otherwise be unavailable to the ultraviolet light source (not shown).

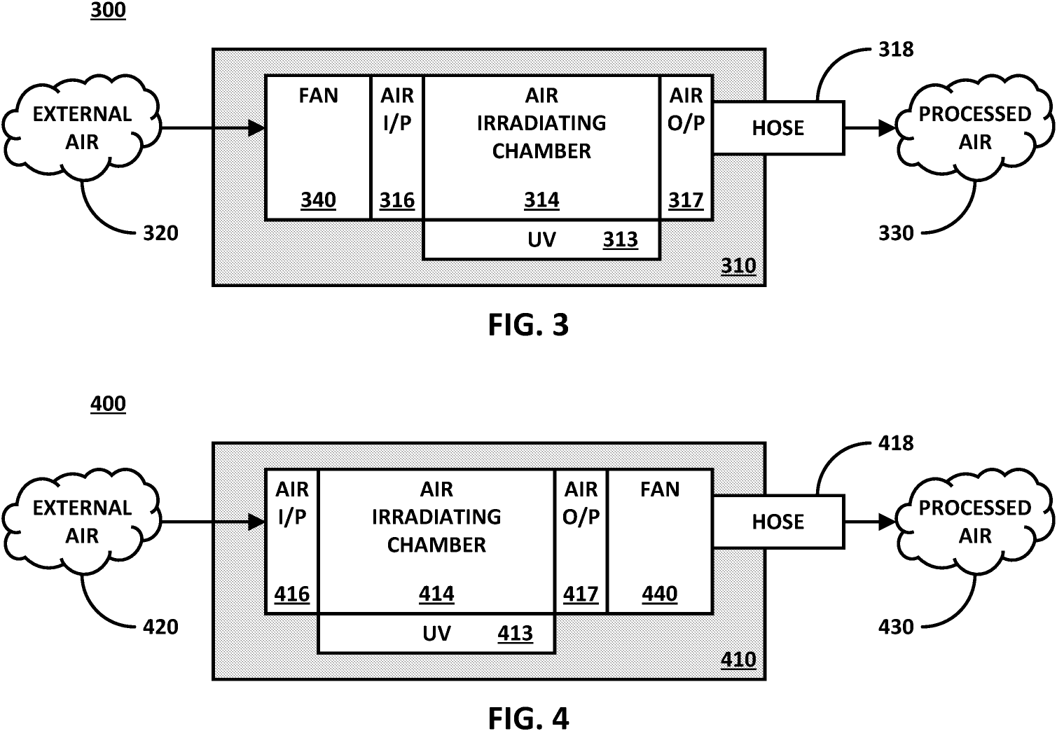

Referring to FIG. 3, a conceptual diagram of a system employing a user-wearable breathing apparatus in accordance with an embodiment of the disclosure is shown. System 300 comprises apparatus 310, which further comprises ultraviolet light source 313, air irradiating chamber 314, air input 316, air output 317, hose 318, and fan 340. Certain components which may be present in various embodiments of apparatus 310, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Ultraviolet light source 313 and air irradiating chamber 314 may be coupled to apparatus 310 such that ultraviolet light from ultraviolet light source 313 may irradiate air passing through air irradiating chamber 314. In some embodiments, ultraviolet light source 313 may be disposed substantially in parallel with air irradiating chamber 314 and irradiating air irradiating chamber 314 substantially perpendicular to the airflow. Air irradiating chamber 314 may further comprise air input 316 and air output 317. Otherwise, ultraviolet light source 313 and air irradiating chamber 314 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2A. Air input 316 may be coupled to a fan 340. A first end of a hose 318 may be coupled to the air output 317. A second end of hose 318 may be configured to couple to a head-wearable breathing assembly (not shown). Fan 340 may be coupled to a power supply (not shown). In some embodiments, it may be the same power supply (not shown) as for ultraviolet light source 313. In other embodiments, it may be a different power supply (not shown).

In operation, system 300 may draw in external air 320 through fan 340, air input 316, and into air irradiating chamber 314. Air irradiating chamber 314 may be ultra-violet transparent. As the air traverses the air irradiating chamber 314, it may be irradiated by ultraviolet light source 313. The intensity of the ultraviolet light from ultraviolet light source 113 may be greater than or substantially equal to 50 micro-Watts per square centimeter (µW/cm²), and its wavelength may be between substantially 100 nanometers and substantially 280 nanometers. Fan 340 may move air out of air irradiating chamber 314 through air output 317 and into hose 318. The processed air 330 may be directed to a user (not shown) for breathing. Although fan 340 is referred to as a “fan” for clarity of presentation, for purposes of this disclosure, the term “fan” may be taken to mean any similar air moving device, like, for example, a fan, an air pump, a propeller, an impeller, etc.

Referring to FIG. 4, a conceptual diagram of a system employing a user-wearable breathing apparatus in accordance with an embodiment of the disclosure is shown. System 400 comprises apparatus 410, which further comprises ultraviolet light source 413, air irradiating chamber 414, air input 416, air output 417, hose 418, and fan 440. Certain components which may be present in various embodiments of apparatus 410, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Ultraviolet light source 413 and air irradiating chamber 414 may be coupled to apparatus 410 such that ultraviolet light from ultraviolet light source 413 may irradiate air passing through air irradiating chamber 414. In some embodiments, ultraviolet light source 413 may be disposed substantially in parallel with air irradiating chamber 414 and irradiating air irradiating chamber 414 substantially perpendicular to the airflow. Air irradiating chamber 414 may further comprise air input 416 and air output 417. Otherwise, ultraviolet light source 413 and air irradiating chamber 414 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2. A first end of a hose 418 may be coupled to the fan 440. A second end of hose 418 may be configured to couple to a head-wearable breathing assembly (not shown). Fan 440 may be coupled to a power supply (not shown). In some embodiments, it may be the same power supply (not shown) as for ultraviolet light source 413. In other embodiments, it may be a different power supply (not shown).

In operation, system 400 may draw in external air 420 through air input 416 and into air irradiating chamber 414 in response to the operation of fan 440. Air irradiating chamber 414 may be ultra-violet transparent. As the air traverses the air irradiating chamber 414, it may be irradiated by ultraviolet light source 413. The intensity of the ultraviolet light from ultraviolet light source 413 may be greater than or substantially equal to 50 micro-Watts per square centimeter (µW/cm²), and its wavelength may be between substantially 100 nanometers and substantially 280 nanometers. Fan 440 may draw air out of air irradiating chamber 414 through air output 417 and into hose 418. The processed air 430 may be directed to a user (not shown) for breathing. Although fan 440 is referred to as a “fan” for clarity of presentation, for purposes of this disclosure, the term “fan” may be taken to mean any similar air moving device, like, for example, a fan, an air pump, a propeller, an impeller, etc.

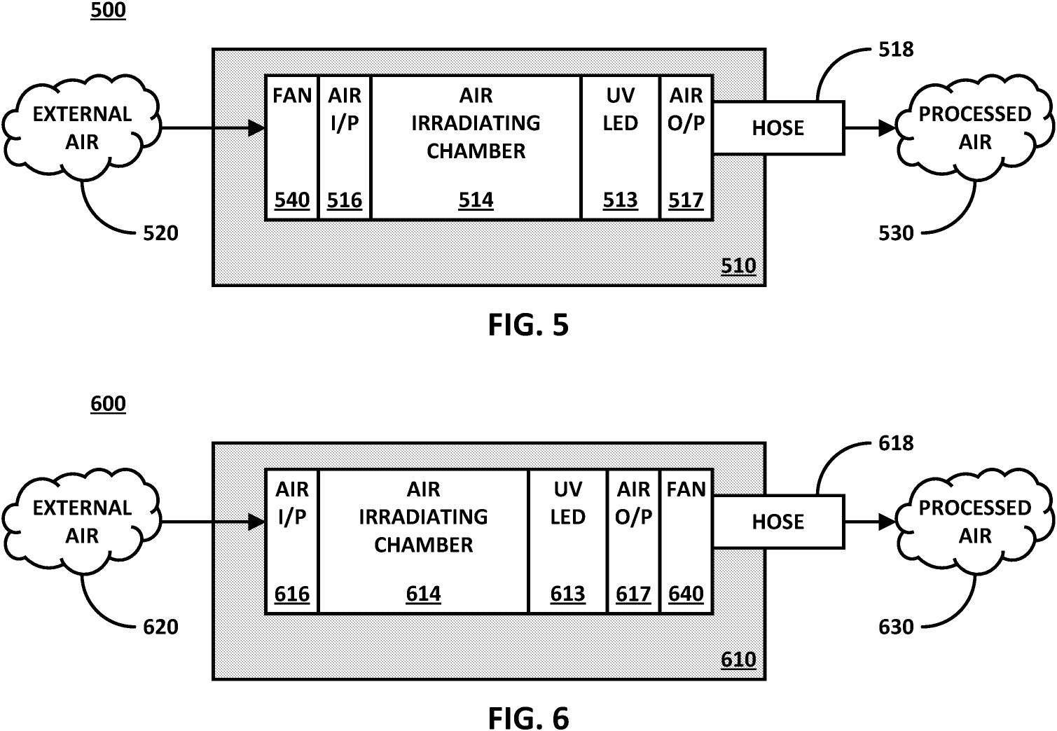

Referring to FIG. 5, a conceptual diagram of a system employing a user-wearable breathing apparatus in accordance with an embodiment of the disclosure is shown. System 500 comprises apparatus 510, which further comprises ultraviolet light source 513, air irradiating chamber 514, air input 516, air output 517, hose 518, and fan 540. Certain components which may be present in various embodiments of apparatus 510, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Ultraviolet light source 513 and air irradiating chamber 514 may be coupled to apparatus 510 such that ultraviolet light from ultraviolet light source 513 may irradiate air passing through air irradiating chamber 514. Air irradiating chamber 514 may further comprise air input 516 and air output 517. In some embodiments, ultraviolet light source 513 may be disposed substantially adjacent to air output 517 and irradiating air irradiating chamber 514 substantially perpendicular to the airflow. In other embodiments, ultraviolet light source 513 may be internal to air irradiating chamber 514. In many embodiments, ultraviolet light source 513 may comprise Light Emitting Diodes (LEDs). In more embodiments, the LEDs may be arranged in a ring. In further embodiments, the ring may be disposed substantially coaxially with air output 517. Otherwise, ultraviolet light source 513 and air irradiating chamber 514 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2.

Air input 516 may be coupled to a fan 540. A first end of the hose 518 may be coupled to the air output 517. A second end of hose 518 may be configured to couple to a head-wearable breathing assembly (not shown). Fan 540 may be coupled to a power supply (not shown). In some embodiments, it may be the same power supply (not shown) as for ultraviolet light source 513. In other embodiments, it may be a different power supply (not shown).

In operation, system 500 may draw in external air 520 through fan 540, air input 516, and into air irradiating chamber 514. Air irradiating chamber 514 may be ultra-violet transparent. As the air traverses the air irradiating chamber 514, it may be irradiated by ultraviolet light source 513. The intensity of the ultraviolet light from ultraviolet light source 513 may be greater than or substantially equal to 50 micro-Watts per square centimeter (µW/cm²), and its wavelength may be between substantially 100 nanometers and substantially 280 nanometers. Fan 540 may move air out of air irradiating chamber 514 through air output 517 and into hose 518. The processed air 530 may be directed to a user (not shown) for breathing. Although fan 540 is referred to as a “fan” for clarity of presentation, for purposes of this disclosure, the term “fan” may be taken to mean any similar air moving device, like, for example, a fan, an air pump, a propeller, an impeller, etc.

Referring to FIG. 6, a conceptual diagram of a system employing a user-wearable breathing apparatus in accordance with an embodiment of the disclosure is shown. System 600 comprises apparatus 610, which further comprises ultraviolet light source 613, air irradiating chamber 614, air input 616, air output 617, hose 618, and fan 640. Certain components which may be present in various embodiments of apparatus 610, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Ultraviolet light source 613 and air irradiating chamber 614 may be coupled to apparatus 610 such that ultraviolet light from ultraviolet light source 613 may irradiate air passing through air irradiating chamber 614. Air irradiating chamber 614 may further comprise air input 616 and air output 617. In some embodiments, ultraviolet light source 613 may be disposed substantially adjacent to air output 617 and irradiating air irradiating chamber 614 substantially parallel to the airflow. In other embodiments, ultraviolet light source 613 may be internal to air irradiating chamber 614. In yet other embodiments, ultraviolet light source 613 may comprise Light Emitting Diodes (LEDs). In still more embodiments, the LEDs may be arranged in a ring. In further embodiments, the ring may be disposed substantially coaxially with air output 617. Otherwise, ultraviolet light source 613 and air irradiating chamber 614 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2.

In operation, system 600 may draw in external air 620 through air input 616 and into air irradiating chamber 614 in response to the operation of fan 640. Air irradiating chamber 614 may be ultra-violet transparent. As the air traverses the air irradiating chamber 614, it may be irradiated by ultraviolet light source 613. The intensity of the ultraviolet light from ultraviolet light source 113 may be greater than or substantially equal to 50 micro-Watts per square centimeter (µW/cm²), and its wavelength may be between substantially 100 nanometers and substantially 280 nanometers. Fan 640 may draw air out of air irradiating chamber 614 through air output 617 and into hose 618. The processed air 630 may be directed to a user for breathing. Although fan 640 is referred to as a “fan” for clarity of presentation, for purposes of this disclosure, the term “fan” may be taken to mean any similar air moving device, like, for example, a fan, an air pump, a propeller, an impeller, etc.

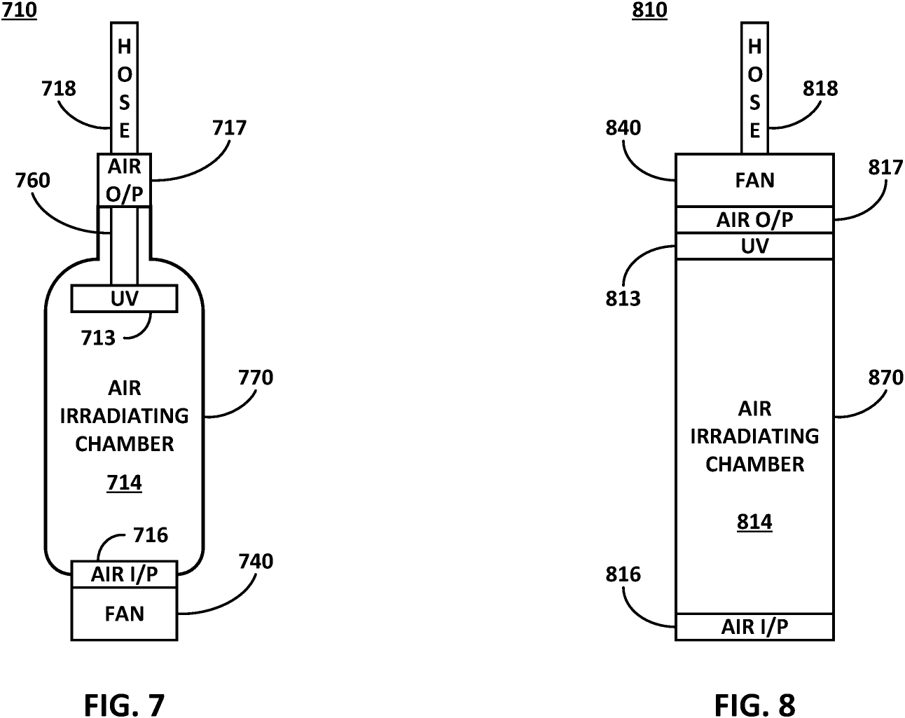

Referring to FIG. 7, a conceptual diagram of a user-wearable breathing apparatus in accordance with an embodiment of the disclosure is shown. Apparatus 710 comprises ultraviolet light source 713, air irradiating chamber 714, air input 716, air output 717, hose 718, and fan 740. Certain components which may be present in various embodiments of apparatus 710, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Air irradiating chamber 714 may comprise a Poly-Ethylene Terephthalate (PET) bottle 770 with a bottom and a neck. In some embodiments, the PET bottle 770 may be painted with an ultraviolet-opaque paint (not shown), while in alternative embodiments, other sorts of ultraviolet-opaque covering substantially enclosing PET bottle 770 may be used, like, for example, a backpack, a bag, a sack, a shoulder bag, a shirt-like garment, an ultraviolet-opaque box, and/or an ultraviolet-opaque container, etc.

Air irradiating chamber 714 may comprise air input 716 and air output 717. In many embodiments, air input 716 may comprise one or more air holes in the bottom of PET bottle 770. The number and diameter of these air holes may be a matter of design choice. In some embodiments, fan 740 may be coupled to air input 716 and configured to draw air into air irradiating chamber 714.

In many embodiments, air output 717 may be coupled to the neck of PET bottle 770 and to a first end of hose 718. A second end of hose 718 may be configured to couple to a head-wearable breathing assembly (not shown). Air output 717 may seal the neck of PET bottle 770 and direct airflow to hose 718. Ultraviolet light source support 760 may have a first end coupled to air output 717 and may also be coupled to ultraviolet light source 713. In some embodiments, ultraviolet light source 713 may comprise LEDs which may be arranged in a ring coaxial to air output 717. In other embodiments, ultraviolet light source 713 and fan 740 may be coupled to a single power supply (not shown). In alternative embodiments, ultraviolet light source 713 and fan 740 may each be coupled to one of two different power supplies (not shown).

Ultraviolet light source 713 and air irradiating chamber 714 may be arranged such that ultraviolet light from ultraviolet light source 713 may irradiate air passing through air irradiating chamber 714. In some embodiments, ultraviolet light source 713 may irradiate air irradiating chamber 714 substantially parallel to the airflow. Otherwise, ultraviolet light source 713 and air irradiating chamber 714 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2 and/or as ultraviolet light source 513 and/or air irradiating chamber 514 as discussed with respect to FIG. 5 and/or as ultraviolet light source 613 and/or air irradiating chamber 614 as discussed with respect to FIG. 6.

In operation, processing external air (not shown), apparatus 710 may draw in external air through fan 740, air input 716, and into air irradiating chamber 714. As the air traverses the air irradiating chamber 714, it may be irradiated by ultraviolet light source 713. The intensity of the ultraviolet light from ultraviolet light source 713 may be greater than or substantially equal to 50 micro-Watts per square centimeter (µW/cm²), and its wavelength may be between substantially 100 nanometers and substantially 280 nanometers. Fan 740 may move air out of air irradiating chamber 714 through air output 717 and into hose 718. The processed air (not shown) may be directed to a user (not shown) for breathing. Although fan 740 is referred to as a “fan” for clarity of presentation, for purposes of this disclosure, the term “fan” may be taken to mean any similar air moving device, like, for example, a fan, an air pump, a propeller, an impeller, etc.

Referring to FIG. 8, a conceptual diagram of a user-wearable breathing apparatus in accordance with an embodiment of the disclosure is shown. Apparatus 810 comprises ultraviolet light source 813, air irradiating chamber 814, air input 816, air output 817, hose 818, and fan 840. Certain components which may be present in various embodiments of apparatus 810, like, for example, a power supply, a power regulator, additional filters, a nitrogen scrubber, an ultraviolet-opaque covering, and optional functions like a processor, a non-volatile memory, a communications transceiver, and an antenna, are not shown to avoid obscuring the inventive concepts disclosed.

Air irradiating chamber 814 may comprise a container 870 with a bottom and an open top. In some embodiments, container 870 may be painted with an ultraviolet-opaque paint (not shown), while in alternative embodiments, other sorts of ultraviolet-opaque covering substantially enclosing container 870 may be used, like, for example, a backpack, a bag, a sack, a shoulder bag, a shirt-like garment, an ultraviolet-opaque box, and/or an ultraviolet-opaque container, etc.

Air irradiating chamber 814 may comprise air input 816 and air output 817. In many embodiments, air input 816 may comprise one or air more holes in the bottom of container 870. The number and diameter of these air holes may be a matter of design choice. In many other embodiments, air output 817 may comprise the open top of container 870. A first end of hose 818 may be coupled to fan 840. In some other embodiments, fan 840 may seal air output 817 to draw air through air input 816, through air irradiating chamber 814, through fan 840, and into hose 818. A second end of hose 818 may be configured to couple to a head-wearable breathing assembly (not shown). In some embodiments, ultraviolet light source 813 may comprise LEDs which may be arranged in a ring coaxial to air output 817. In other embodiments, ultraviolet light source 813 and fan 840 may be coupled to a single power supply (not shown). In alternative embodiments, ultraviolet light source 813 and fan 840 may each be coupled to one of two different power supplies (not shown).

Ultraviolet light source 813 and air irradiating chamber 814 may be arranged such that ultraviolet light from ultraviolet light source 813 may irradiate air passing through air irradiating chamber 814. In some embodiments, ultraviolet light source 813 may irradiate the air traversing air irradiating chamber 814 substantially parallel to the airflow. Otherwise, ultraviolet light source 813 and air irradiating chamber 814 may operate in a similar manner as ultraviolet light source 113 and/or air irradiating chamber 114 as discussed with respect to FIG. 1 and/or as ultraviolet light source 213 and/or air irradiating chamber 214 as discussed with respect to FIG. 2 and/or as ultraviolet light source 513 and/or air irradiating chamber 514 as discussed with respect to FIG. 5 and/or as ultraviolet light source 613 and/or air irradiating chamber 614 as discussed with respect to FIG. 6.

In operation, apparatus 810 may draw in external air through air input 816 and into air irradiating chamber 814. As the air traverses the air irradiating chamber 814, it may be irradiated by ultraviolet light source 813. The intensity of the ultraviolet light from ultraviolet light source 813 may be greater than or substantially equal to 50 micro-Watts per square centimeter (µW/cm²), and its wavelength may be between substantially 100 nanometers and substantially 280 nanometers. Fan 840 may draw air out of air irradiating chamber 814 and into hose 818. The processed air (not shown) may be directed to a user (not shown) for breathing. Although fan 840 is referred to as a “fan” for clarity of presentation, for purposes of this disclosure, the term “fan” may be taken to mean any similar air moving device, like, for example, a fan, an air pump, a propeller, an impeller, etc.

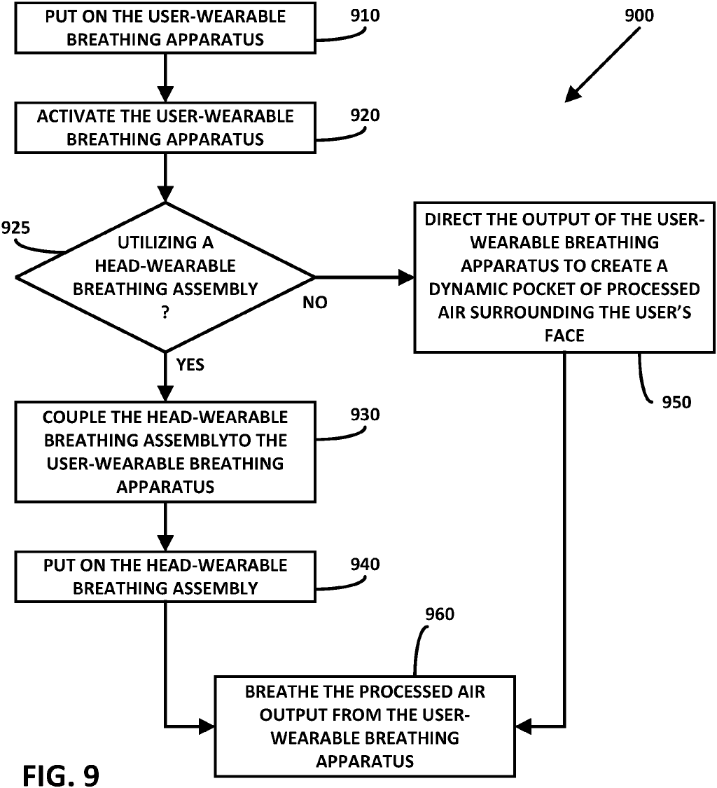

Referring to FIG. 9, a flowchart depicting a process 900 for operating a user-wearable breathing apparatus in accordance with an embodiment of the disclosure is shown. Process 900 may begin with a user putting on and wearing the user-wearable breathing apparatus (block 910). In different embodiments, this may take the form of putting a backpack on the user’s back, hanging a carrying bag or container from a strap attached over the user’s shoulder, coupling a bag, a sack, a shoulder bag, or container to the user’s belt, etc. In some embodiments, the user-wearable breathing apparatus may be coupled to the user’s upper torso with straps around the user’s upper torso. In alternative embodiments, a shirt-like garment configured to hold the user-wearable breathing apparatus to the users’s upper torso may be worn.

The user may activate (or power up or turn on) the user-wearable breathing apparatus (block 920). The user may then determine if a head-wearable breathing assembly is to be utilized (block 925). Such an assembly may be a CPAP mask, a silhouette nasal mask, a dental nitrous oxide mask, or the like. If a head-wearable breathing assembly is to be utilized, the user may couple the head-wearable breathing assembly to the user-wearable breathing apparatus (block 930) and may put on the head-wearable breathing assembly (block 940). The user may proceed to breathe the processed air from the user-wearable breathing apparatus (block 960).

If a head-wearable breathing assembly is not to be utilized, the user may direct the output of the user-wearable breathing apparatus to create a dynamic pocket of processed air surrounding a user’s face (block 950). This may involve pointing the output to direct a steady stream of processed air to the user’s face. This may take the form, for example, of a stiff but flexible hose being employed to direct the air to the desired location. The user may proceed to breathe the processed air from the user-wearable breathing apparatus (block 960).

It is understood that there may be a great many different orders in which the above actions may be taken. In some cases, two steps may be performed concurrently, in whole or in part, or even performed in a different order altogether than described above.

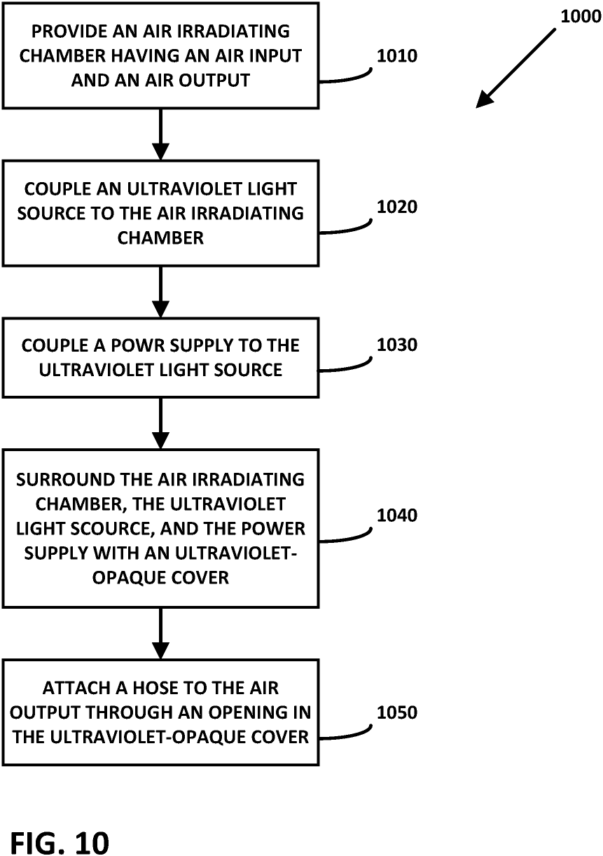

Referring to FIG. 10, a flowchart depicting a process 1000 for constructing a user-wearable breathing apparatus in accordance with an embodiment of the disclosure is shown. Process 1000 may begin with providing an air irradiating chamber having an air input and an air output (block 1010). The air irradiating chamber may delay the time needed for the air to traverse it. In some embodiments, this may be accomplished with an indirect maze-like structure, by filling the air irradiating chamber with a densely packed fibrous material, or by filling the air irradiating chamber with a pours material, or the like. Anti-viral materials like, for example, Zoono, graphene, etc., or anti-microbial materials like, for example, activated charcoal or nano-silver may be included in whole or in part. In alternative embodiments, the chamber may be empty except for the air traversing it.

An ultraviolet light source may be coupled to the air irradiating chamber (block 1020). The ultraviolet light source may be configured to irradiate the air inside the air irradiating chamber with ultraviolet light. Ultraviolet light may kill viruses. Higher intensities and longer exposure times being may be more effective. In some embodiments, the ultraviolet light source may irradiate the air in the air irradiating chamber from outside if the air irradiating chamber is constructed with ultraviolet-transparent materials. In other embodiments, the ultraviolet light source may be located internal to the air irradiating chamber.

A power supply may be coupled to the ultraviolet light source (block 1030). The ultraviolet light source may require power. In many embodiments, an air moving device like, for example, a fan, an air pump, a propeller, an impeller, etc., may be present. In some embodiments, the fan may share the power supply with the ultraviolet light source, while in alternate embodiments, the fan may require a separate power supply. For purposes of this disclosure, the term “fan” may be taken to mean any similar air moving device, like, for example, a fan, an air pump, a propeller, an impeller, etc.

The air irradiating chamber, the ultraviolet light source, and the power supply may be surrounded by an ultraviolet-opaque cover (block 1040). In some embodiments, this ultraviolet-opaque cover may protect the user and others from ultraviolet light from the ultraviolet light source. In some other embodiments, the ultraviolet-opaque cover can serve as a filter for air being drawn into the air input of the air irradiating chamber if constructed from cloth or some other porous material. Anti-viral materials like, for example, Zoono, graphene, etc., or anti-microbial materials like, for example, activated charcoal or nano-silver, etc., may be used to treat the ultraviolet-opaque cover material. In still other embodiments, the ultraviolet-opaque cover may provide an anchor for straps, loops, hooks, or the like, needed for the user to wear the user-wearable breathing apparatus.

A hose may be attached to the air output through an opening in the ultraviolet-opaque cover (block 1050). In some embodiments, the hose may be configured to couple to a head-wearable breathing assembly, and a longer hose with significant strength and flexibility may be required. In other embodiments, the hose is used to direct the processed air to create a dynamic pocket of processed air surrounding the user’s face, and a shorter hose with significant rigidity may be required.

It is understood that there may be a great many different orders in which the above actions may be taken. In some cases, two steps may be performed concurrently, in whole or in part, or even performed in a different order altogether than described above.

Information as herein shown and described in detail is fully capable of attaining the above-described object of the present disclosure, the presently preferred embodiment of the present disclosure, and is, thus, representative of the subj ect matter that is broadly contemplated by the present disclosure. The scope of the present disclosure fully encompasses other embodiments that might become obvious to those skilled in the art, and is to be limited, accordingly, by nothing other than the appended claims. Any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for solutions to such problems to be encompassed by the present claims. Furthermore, no element, component, or method step in the present disclosure is intended to be dedicated to the public regardless of whether the element, component, or method step is explicitly recited in the claims. Various changes and modifications in form, material, work-piece, and fabrication material detail can be made, without departing from the spirit and scope of the present disclosure, as set forth in the appended claims, as might be apparent to those of ordinary skill in the art, are also encompassed by the present disclosure.

What is claimed is:

1. A user-wearable breathing apparatus, comprising:
an air irradiating chamber having an air input and an air output;
an ultraviolet light source coupled to the air irradiating chamber and configured to irradiate the air irradiating chamber with ultraviolet light;
a power supply coupled to the ultraviolet light source;
an ultraviolet-opaque cover with an opening substantially surrounding the user-wearable breathing apparatus; and
a first hose with a first end coupled to the air output, wherein:
the first hose passes through the opening, and
a second end of the first hose is disposed outside the ultraviolet-opaque cover.

2. The user-wearable breathing apparatus of claim 1, wherein the air irradiating chamber further comprises:
a first one-way valve coupled between the ultraviolet-opaque covering and the air input;
a second one-way valve coupled between the air output and the first hose; and wherein:
the apparatus is configured to be coupled to the upper torso of a user,
the apparatus is configured so that the breathing motion of the user draws air into the air input and through the first one-way valve into the air irradiating chamber, and
the apparatus is configured so that the breathing motion of the user draws the air out of the air irradiating chamber through the second one-way valve.

3. The user-wearable breathing apparatus of claim 2, wherein the ultraviolet-opaque cover is selected from the group consisting of: a backpack, a bag, a sack, a shoulder bag, a shirt-like garment, an ultraviolet-opaque box, an ultraviolet-opaque container, and ultraviolet-opaque paint.

4. The user-wearable breathing apparatus of claim 3, wherein:
the intensity of the ultraviolet light is greater than or substantially equal to 50 microWatts per square centimeter (µW/cm2); and
the wavelength of the ultraviolet light is between substantially 100 nanometers and substantially 280 nanometers.

5. The user-wearable breathing apparatus of claim 4, further comprising an air filter coupled to the air input, wherein the air filter comprises at least in part an anti-viral material.

6. The user-wearable breathing apparatus of claim 4, further comprising an air filter coupled to the air output, wherein the air filter comprises at least in part an anti-viral material.

7. The user-wearable breathing apparatus of claim 1, wherein:
the intensity of the ultraviolet light is greater than or substantially equal to 50 microWatts per square centimeter (µW/cm2); and
the wavelength of the ultraviolet light is between substantially 100 nanometers and substantially 280 nanometers.

8. The user-wearable breathing apparatus of claim 7, wherein the ultraviolet-opaque cover is selected from the group consisting of: a backpack, a bag, a sack, a shoulder bag, a shirt-like garment, an ultraviolet-opaque box, an ultraviolet-opaque container, and ultraviolet-opaque paint.

9. The user-wearable breathing apparatus of claim 8, further comprising an air filter coupled to the air input, wherein the air filter comprises at least in part an anti-viral material.

10. The user-wearable breathing apparatus of claim 8, further comprising an air filter coupled to the air output, wherein the air filter comprises at least in part an anti-viral material.

11. The user-wearable breathing apparatus of claim 8, further comprising an air moving device coupled to the air input, wherein the air moving device is configured to move air into the air irradiating chamber through the air input.

12. The user-wearable breathing apparatus of claim 8, further comprising an air moving device coupled between the air output and the first end of the first hose, wherein the air moving device is configured to move air into the air irradiating chamber through the air input.

13. The user-wearable breathing apparatus of claim 8, wherein the ultraviolet light source comprises one or more light emitting diodes.

14. The user-wearable breathing apparatus of claim 8, further comprising a nitrogen scrubber coupled to the air irradiating chamber.

15. The user-wearable breathing apparatus of claim 8, further comprising:
a power regulator coupled to the power supply; and
an energy storage device.

16. The user-wearable breathing apparatus of claim 15, wherein:
the energy storage device is rechargeable;
the energy storage device is selected from the group consisting of: a battery, a capacitor, and a super-capacitor;
the power regulator is configured to couple to an external source of energy; and
the external source of energy is selected from the group consisting of: a power main, a battery charging device, a solar panel, a kinetic-to-electrical energy converter, an ambient radiation-to-electrical energy converter, and a temperature-difference-to-electrical energy converter.

17. The user-wearable breathing apparatus of claim 8, wherein:
the air irradiating chamber further comprises a gas port; and
the gas port is configured to couple to a second hose.

18. The user-wearable breathing apparatus of claim 12, wherein the air moving device is configured to create a negative pressure from the user-wearable breathing apparatus to a user coupled to the second end of the first hose.

19. The user-wearable breathing apparatus of claim 7, wherein the air irradiating chamber further comprises:
a Poly-Ethylene Terephthalate (PET) bottle having a bottom and a neck; and
an air moving device coupled between the air input and the ultraviolet-opaque cover, wherein:
the air moving device is configured to move air into the air irradiating chamber through the air input,
the air input comprises at least one air opening in the bottom of the PET bottle,
the air output is coupled to the neck of the PET bottle, and
the ultraviolet light source is coupled to the air output.

20. The user-wearable breathing apparatus of claim 7, wherein:
the air irradiating chamber further comprises a container with a bottom and an open top;
an air moving device is coupled between the open top and the first hose; and wherein:
the air output is the open top of the container,
the air moving device is configured to move air from the air irradiating chamber through the air output and into the first hose,
the air input comprises at least one air opening through the bottom of the container, and
the ultraviolet light source is coupled to the air output.

21. The user-wearable breathing apparatus of claim 15, further comprising a processor coupled to the power regulator and the power supply, wherein:
the processor executes machine instructions stored in a non-volatile machine-readable memory,
the processor manages the power regulator and power supply, and
the processor monitors the operational state of the user-wearable breathing apparatus.

22. The user-wearable breathing apparatus of claim 21, further comprising a communications transceiver coupled to the processor and the power supply, wherein:
the communications transceiver is configured to communicate with an external system,
the processor is configured to communicate with the external system through the communications transceiver,
the processor is configured to report on the operational state of user-wearable breathing apparatus, and
the processor is configured to update the machine instructions stored in the non-volatile machine-readable memory.

23. A method of operating a user-wearable breathing apparatus for processing external air into processed air, the user-wearable breathing apparatus comprising an ultraviolet light source coupled to an air irradiating chamber and a power supply, an ultraviolet-opaque cover coupled to the air irradiating chamber, and a hose coupled to the air irradiating chamber and disposed to pass through a hole in the ultraviolet-opaque cover, the method comprising:
putting on the user-wearable breathing apparatus; and
activating the user-wearable breathing apparatus.

24. The method of operating a user-wearable breathing apparatus for processing external air into processed air of claim 23, further comprising:
coupling a head-wearable breathing assembly to the user-wearable breathing apparatus; and
putting on the head-wearable breathing assembly.

25. The method of claim 24, wherein the hose is retractable and configured to couple to a direct oxygen delivery system.

26. The method of operating a user-wearable breathing apparatus for processing external air into processed air of claim 23, further comprising directing the output of the user-wearable breathing apparatus to create a dynamic pocket of processed air surrounding the face of a user.

27. A method of constructing a user-wearable breathing apparatus for processing external air into processed air, the method comprising:
providing an air irradiating chamber having an air input and an air output;
coupling an ultraviolet light source to the air irradiating chamber and configuring the ultraviolet light source to irradiate the air irradiating chamber with ultraviolet light;
coupling a power supply to the ultraviolet light source;
surrounding the air irradiating chamber, the ultraviolet light source, and the power supply with an ultraviolet-opaque cover; and
attaching a hose to the air output through an opening in the ultraviolet-opaque cover.
